(19) United States
(12) Patent Application Publication
(10) Pub. No.: US 2017/0214786 A1
(43) Pub. Date: Jul. 27, 2017

(54) ELECTRONIC DEVICE AND METHOD THEREOF BASED ON MOTION RECOGNITION

(71) Applicant: Samsung Electronics Co., Ltd., Gyeonggi-do (KR)

(72) Inventors: Seungjai Lee, Seongnam-si (KR); Yeongju Kim, Suwon-si (KR); Sanggon Shin, Hwaseong-si (KR)

(21) Appl. No.: 15/399,498

(22) Filed: Jan. 5, 2017

(30) Foreign Application Priority Data
Jan. 26, 2016 (KR) 10-2016-0009420

(51) Int. Cl. H04M 1/725; A61B 5/024; A61B 5/00; H04M 1/03

(52) U.S. Cl. CPC H04M 1/72569; H04M 1/7253; H04M 1/03; A61B 5/02438; A61B 5/6815; A61B 5/7285; A61B 2562/0219

(57) ABSTRACT

An electronic device and a control method thereof based on motion recognition. The electronic device receives at least one signal through a communication unit thereof and, based on the received signal, executes a vibration notification. Also, the device detects a motion thereof through at least one motion sensor equipped therein and determines whether a state of at least one connection unit equipped in a housing is changed. The device also obtains information associated with a user's body part on which the device is worn, based on at least one of information associated with the detected motion and information associated with the changed state of the connection unit. Then, based on the obtained information, the device performs at least one function.

### CROSS-REFERENCE TO RELATED APPLICATION(S) AND CLAIM OF PRIORITY

The present application is related to and claims the priority under 35 U.S.C. §119(a) of Korean patent application filed on Jan. 26, 2016 in the Korean intellectual property office and assigned serial number 10-2016-0009420, the entire disclosure of which is hereby incorporated by reference.

### TECHNICAL FIELD

Embodiments of the present disclosure relate to an electronic device and a control method thereof based on motion recognition.

### BACKGROUND

With the development of technologies related to an electronic device, a great variety of services for users are increasingly used in many fields. For example, the electronic device not only has a traditional call function, but also supports various functions such as webpage browsing, content playback, a social networking service (SNS), a digital camera, and the like.

Further, recent information communication technologies make it possible to use even ordinary things, in addition to a personal computer (PC) or a smart phone, as information communication terminals. Particularly, various kinds of wearable devices are now attracting attention in the art. While initial forms of wearable devices were developed for specific purposes or targeted at limited markets, recent wearable devices are developed for more ordinary purposes in the form of being available for normal consumers. Beyond simple accessories, recent wearable devices come to realize high-tech electronic devices capable of interacting with a user at the closest position to a user's body.

The wearable device can collect detailed information about surrounding environments or about variations in user's body continuously in real time. Also, the wearable device can provide a new user experience through a voice recognition function, a gesture recognition function, a healthcare function, etc. by interacting with a mobile device. Having various advantages, the wearable device is now rapidly popularized and also regarded as a new fashion item.

By the way, most wearable devices having no earphone function need to be fixed near a user's mouth during a voice call. Unfortunately, this may cause a user any inconvenience. Further, other party's voice may spread through a speaker equipped in the wearable device, thus incurring an inadequate protection of privacy.

### SUMMARY

To address the above-discussed deficiencies, it is a primary object to provide a user interaction at an electronic device may include operations of receiving at least one signal through a communication unit of the device; executing a vibration notification, based on the received signal; detecting a motion of the device through at least one motion sensor of the device; determining whether a state of at least one connection unit equipped in a housing of the device is changed; obtaining information associated with a user's body part on which the device is worn, based on at least one of information associated with the detected motion and information associated with the changed state of the connection unit; and performing at least one function, based on the obtained information.

Additionally, according to various embodiments of the present disclosure, an electronic device may include a flexible housing including at least one connection unit; a communication unit located in the housing; at least one vibration motor located in the housing; at least one motion sensor location in the housing; a processor electrically coupled to at least one of the communication unit, the at least one vibration motor, and the at least one motion sensor; and a memory electrically coupled to the processor. In particular, the memory may store instructions causing, when executed, the processor to receive at least one signal through the communication unit, to execute a vibration notification, based on the received signal, to detect a motion of the device through the at least one motion sensor, to determine whether a state of the at least one connection unit is changed, to obtain information associated with a user's body part on which the device is worn, based on at least one of information associated with the detected motion and information associated with the changed state of the connection unit, and to perform at least one function, based on the obtained information.

According to various embodiments, the electronic device disclosed herein can be used at any time as an earring capable of performing an earphone function without a need to wear it on the ear or around the neck.

Also, according to various embodiments, the electronic device and related method disclosed herein can satisfy both portability and aesthetic requisite by means of a bracelet-transformable form of the device.

And also, according to various embodiments, the electronic device is extendable and retractable in length and thus can be customized depending on users.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a network environment including an electronic device according to various embodiments.

FIG. 2 illustrates an electronic device according to various embodiments.

FIG. 3 illustrates a program module according to various embodiments.

FIG. 4 illustrates a method for controlling a device by recognizing a motion of the device according to various embodiments.

FIGS. 5A and 5B illustrate a structure of an electronic device according to various embodiments.

FIG. 6 illustrates a connection unit of an electronic device according to various embodiments.

FIG. 7 illustrates an operation method of an electronic device according to various embodiments.

FIG. 8 illustrates an example of using an electronic device according to various embodiments.

### DETAILED DESCRIPTION

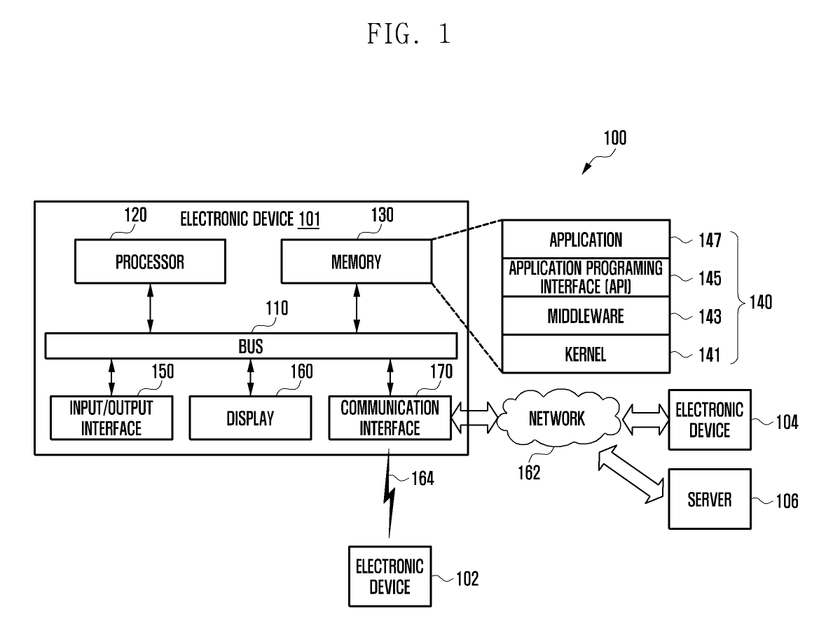

FIGS. 1 through 8, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged electronic device.

The following description, with reference to the accompanying drawings, is provided to assist in a comprehensive understanding of an embodiment of the present disclosure as defined by the claims and their equivalents. It includes various specific details to assist in that understanding but are to be regarded as merely examples. Accordingly, those of ordinary skill in the art will recognize that various changes and modifications of the embodiments described herein may be made without departing from the scope and spirit of the present disclosure. In addition, descriptions of well-known functions and constructions may be omitted for clarity and conciseness.

Expressions such as “include” and “may include” which may be used in the present disclosure denote the presence of the disclosed functions, operations, and constituent elements and do not limit one or more additional functions, operations, and constituent elements. In the present disclosure, terms such as “include” and/or “have” may be construed to denote a certain characteristic, number, step, operation, constituent element, component or a combination thereof, but may not be construed to exclude the existence of, or a possibility of, the addition of one or more other characteristics, numbers, steps, operations, constituent elements, components or combinations thereof.

Furthermore, in the present disclosure, the expression “and/or” includes any and all combinations of the associated listed words. For example, the expression “A and/or B” may include A, may include B, or may include both A and B.

In the present disclosure, expressions including ordinal numbers, such as “first” and “second,” etc., may modify various elements. However, such elements are not limited by the above expressions. For example, the above expressions do not limit the sequence and/or importance of the elements. The above expressions are used merely for the purpose to distinguish an element from the other elements. For example, a first user device and a second user device indicate different user devices although both of them are user devices. For example, a first element may be referred to as a second element, and similarly, a second element may also be referred to as a first element without departing from the scope of the present disclosure.

In the case where a component is referred to as being “connected” to, or “accessed” by another component, it should be understood that not only is the component directly connected to or accessed by the other component, but there may also exist another component between them. Meanwhile, in the case where a component is referred to as being “directly connected” or “directly accessed” to another component, it should be understood that there is no component therebetween.

The terms used in the present disclosure are only used to describe specific embodiments, and do not limit the present disclosure. As used herein, the singular forms are intended to include the plural forms as well, unless the context clearly indicates otherwise.

In this disclosure, an electronic device may be a device that involves a communication function. For example, an electronic device may be a mobile phone, or a wearable device (e.g., a head-mounted device (HMD)) such as electronic eyeglasses, electronic clothes, an electronic bracelet, an electronic necklace, an electronic appcessory, or a smart watch), or the like, but is not limited thereto.

Other embodiments of the electronic device include various medical devices (for example, various kinds of portable medical measuring device (blood glucose meter, heart rate meter, blood pressure meter, or a temperature measuring instrument, etc.), global positioning system receiver (GPS) receiver, or the like, but is not limited thereto.

The electronic device may be one or more combinations of the various devices described above. The electronic device may be a flexible electronic device. In addition, an electronic device is not limited to the above-described device, and may include a new electronic device, in accordance with new technological developments. In this document, the term user refers to a human or an electronic device using the electronic device (for example, an artificial intelligence electronic device).

FIG. 1 illustrates an example network environment 100 including an electronic device 101 in accordance with an example embodiment of the present disclosure. Referring to FIG. 1, the electronic device 101 includes a bus 110, a processor 120, a memory 130, an input/output interface (e.g., including input/output circuitry) 150, a display 160, and a communication interface (e.g., including communication circuitry) 170.

The bus 110 may be a circuit designed for connecting the above-discussed elements and communicating data (e.g., a control message) between such elements.

The processor 120 may receive commands from the other elements (e.g., the memory 130, the input/output interface 150, the display 160, or the communication interface 170, etc.) through the bus 110, interpret the received commands, and perform arithmetic or data processing based on the interpreted commands.

The memory 130 may store therein commands or data received from or created at the processor 120 or other elements (e.g., the input/output interface 150, the display 160, or the communication interface 170, etc.). The memory 130 may include programming modules 140 such as a kernel 141, a middleware 143, an application programming interface (API) 145, and an application 147. Each of the programming modules may be composed of software, firmware, hardware, and any combination thereof.

The kernel 141 may control or manage system resources (e.g., the bus 110, the processor 120, the memory 130, etc.) used to execute operations or functions implemented by other programming modules (e.g., the middleware 143, the API 145, and the application 147). Also, the kernel 141 may provide an interface capable of accessing and controlling or managing the individual elements of the electronic device 101 by using the middleware 143, the API 145, or the application 147.

The middleware 143 may serve to go between the API 145 or the application 147 and the kernel 141 in such a manner that the API 145 or the application 147 communicates with the kernel 141 and exchanges data therewith. Also, in relation to work requests received from one or more applications 147 and/or the middleware 143, for example, may perform load balancing of the work requests by using a method of assigning a priority, in which system resources (e.g., the bus 110, the processor 120, the memory 130, etc.) of the electronic device 101 may be used by one or more applications 147.

The API 145 is an interface through which the application 147 is capable of controlling a function provided by the kernel 141 or the middleware 143, and may include, for example, at least one interface or function for file control, window control, image processing, character control, and the like.

The input/output interface 150 may include various input/output circuitry that deliver commands or data, entered by a user through an input/output unit (e.g., circuitry that may include, for example, and without limitation, a sensor, a keyboard, or a touch screen, or the like), to the processor 120, the memory 130, or the communication interface 170 via the bus 110.

The display (e.g., display “module”) 160 may include, for example, a liquid crystal display (LCD), a light emitting diode (LED) display, an organic LED (OLED) display, a micro electro mechanical system (MEMS) display, or an electronic paper display. The display 160 may display various types of content (e.g., text, images, videos, icons, or symbols) for users. The display module 160 may include a touch screen, and may receive, for example, a touch, gesture, proximity, or hovering input by using an electronic device or a part of the user's body.

The communication interface 170 may include various communication circuitry configured to perform communication between the electronic device 101 and the electronic device 104 or the server 106. For example, the communication interface 170 may communicate with any external device by being connected with a network 162 through a wired or wireless communication or via a wireless connection 164.

The wireless communication may include, but not limited to, at least one of WIFI®, BLUETOOTH® (BT), near field communication (NFC), global navigation satellite system (GNSS), or a cellular communication (e.g., machine type communications (MTC), fifth generation (5G), long term evolution (LTE), long term evolution advanced (LTE-A), code division multiple access (CDMA), wideband code division multiple access (WCDMA), universal mobile telecommunications system (UMTS), wireless broadband (WiBro), global system for mobile communications (GSM), etc.). The GNSS may include at least one of global positioning system (GPS), global navigation satellite system (GLONASS), Beidou navigation satellite system (BEIDOU), Galileo, or the European global satellite-based navigation system. Hereinafter, the terms “GPS” and the “GNSS” may be interchangeably used herein. The wired communication may include, at least one of universal serial bus (USB), high definition multimedia interface (HDMI), RS-232 (recommended standard 232), or plain old telephone service (POTS). The network 162 includes, as a telecommunications network at least one of a computer network (e.g., local area network (LAN) or wide area network (WAN)), the Internet, and a telephone network.

The types of the first and second external electronic devices 102 and 104 may be the same as or different from the type of the electronic device 101. The server 106 may include a group of one or more servers. A portion or all of operations performed in the electronic device 101 may be performed in one or more other electronic devices 102, 104 or the server 106. In the case where the electronic device 101 peforms a certain function or service automatically or in response to a request, the electronic device 101 may request at least a portion of functions related to the function or service from another electronic device 102, 104 or the server 106 instead of, or in addition to, performing the function or service for itself. The other electronic device 102, 104 or the server 106 may perform the requested function or additional function, and may transfer a result of the performance to the electronic device 101. The electronic device 101 may additionally process the received result to provide the requested function or service. To this end, for example, a cloud computing technology, a distributed computing technology, or a client-server computing technology may be used.

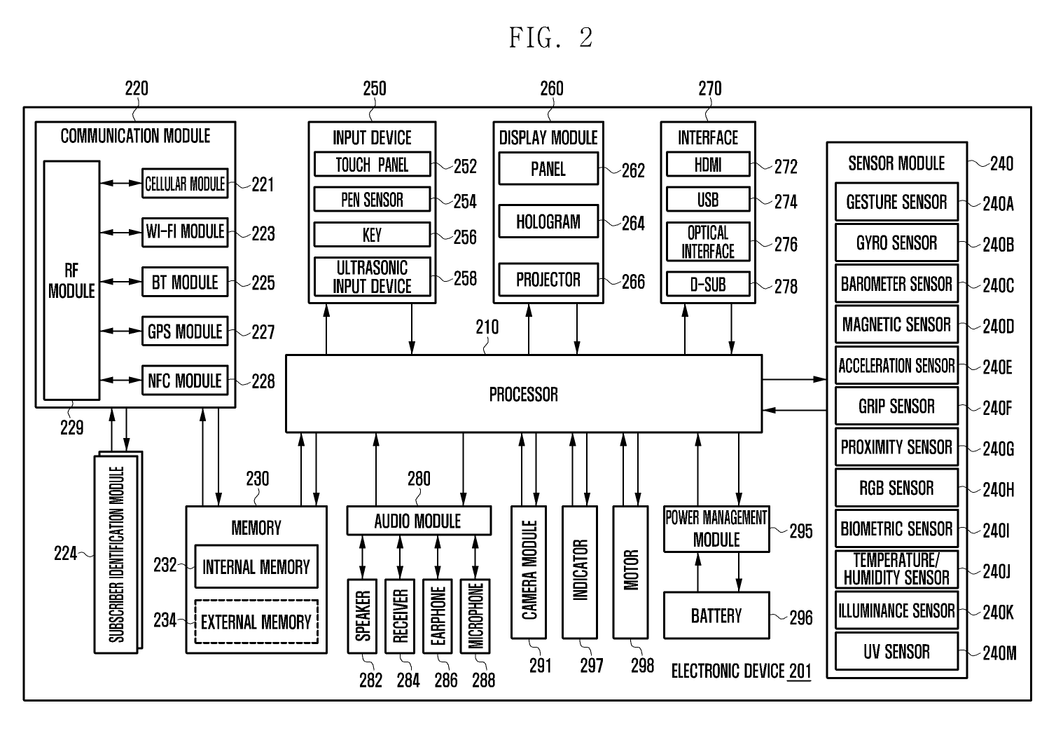

FIG. 2 illustrates an example electronic device 201 in accordance with an example embodiment of the present disclosure. The electronic device 201 may form, for example, the whole or part of the electronic device 101 illustrate in FIG. 1. Referring to FIG. 2, the electronic device 201 includes at least one application processor (AP) 210, a communication module (e.g., including communication circuitry) 220, a subscriber identification module (SIM) card 224, a memory 230, a sensor module 240, an input unit (e.g., including input circuitry) 250, a display 260, an interface (e.g., including interface circuitry) 270, an audio module 280, a camera module 291, a power management module 295, a battery 296, an indicator 297, and a motor 298.

The AP 210 may drive an operating system or applications, control a plurality of hardware or software components connected thereto, and also perform processing and operation for various data including multimedia data. The AP 210 may be formed of a system-on-chip (SoC), for example. According to an embodiment of the present disclosure, the AP 210 may further include a graphic processing unit (GPU).

The communication module 220 (e.g., the communication interface 170) may include various communication circuitry configured to perform data communication with the electronic device 104 or the server 106 connected to the electronic device 201 (e.g., the electronic device 101) through the network. According to an embodiment of the present disclosure, the communication module 220 may include various communication circuitry, such as, for example, and without limitation, a cellular module 221, a WiFi module 223, a BT module 225, a GNSS module 227, an NFC module 228, and an RF (radio frequency) module 229.

The cellular module 221 may offer a voice call, a video call, a message service, an internet service, and the like through a communication network (e.g., machine type communications (MTC), fifth generation (5G), long term evolution (LTE), long term evolution advanced (LTE-A), code division multiple access (CDMA), wideband code division multiple access (WCDMA), universal mobile telecommunications system (UMTS), wireless broadband (WiBro), global system for mobile communications (GSM), WIFI®, BLUETOOTH®, and near field communications (NFC) etc.). Additionally, the cellular module 221 may perform identification and authentication of the electronic device in the communication network, using the SIM card 224. According to an embodiment of the present disclosure, the cellular module 221 may perform at least part of functions the AP 210 may provide. For example, the cellular module 221 may perform at least part of a multimedia control function. Each of the WiFi module 223, the BT module 225, the GNSS module 227 and the NFC module 228 may include a processor for processing data transmitted or received. Although FIG. 2 shows the cellular module 221, the WiFi module 223, the BT module 225, the GNSS module 227 and the NFC module 228 as different blocks, at least part of them may be contained in a single IC (integrated circuit) chip or a single IC package.

The RF module 229 may transmit and receive data, e.g., RF signals or any other electric signals. The RF module 229 may include a transceiver, a PAM (power amp module), a frequency filter, an LNA (low noise amplifier), and the like. Although FIG. 2 shows that the cellular module 221, the WiFi module 223, the BT module 225, the GNSS module 227 and the NFC module 228 share the RF module 229, at least one of them may perform transmission and reception of RF signals through a separate RF module.

The SIM card 224 may include, for example, an embedded SIM including a user identification module, and may include unique identification information (e.g., an integrated circuit card identifier (ICCID)) or subscriber information (e.g., international mobile subscriber identity (IMSI)).

The memory 230 includes an internal memory 232 and an external memory 234. The memory 230 may be, for example, the memory 130 illustrated in FIG. 1. The internal memory 232 may include, for example, at least one of a volatile memory (e.g., a dynamic RAM (DRAM), a static RAM (SRAM), a synchronous dynamic RAM (SDRAM), etc.), and a non-volatile memory (e.g., a one time programmable ROM (OTPROM), a programmable ROM (PROM), an erasable and programmable ROM (EPROM), an electrically erasable and programmable ROM (EEPROM), a mask ROM, a flash ROM, a not AND (NAND) flash memory, a not OR (NOR) flash memory, etc.). According to an embodiment of the present disclosure, the internal memory 232 may be in the form of a solid state drive (SSD). The external memory 234 may further include a flash drive, for example, a compact flash (CF), a secure digital (SD), a micro-secure digital (micro-SD), a mini-secure digital (mini-SD), an extreme digital (xD), a memory stick, and the like. The external memory 234 may be functionally connected to the electronic device 201 through various interfaces.

The sensor module 240 may measure physical quantity or sense an operating status of the electronic device 201, and then convert measured or sensed information into electric signals. The sensor module 240 includes, for example, at least one of a gesture sensor 240A, a gyro sensor 240B, an atmospheric (e.g., barometer or barometric) sensor 240C, a magnetic sensor 240D, an acceleration sensor 240E, a grip sensor 240F, a proximity sensor 240G, a color sensor 240H (e.g., RGB or “red, green, blue” sensor), a biometric sensor 2401, a temperature-humidity sensor 240J, an illumination sensor 240K, and a UV (ultraviolet) sensor 240M. Additionally or alternatively, the sensor module 240 may include, e.g., an E-nose sensor, an EMG (electromyography) sensor, an EEG (electroencephalogram) sensor, an ECG (electrocardiogram) sensor, an IR (infrared) sensor, an iris scan sensor, or a finger scan sensor. Also, the sensor module 240 may include a control circuit for controlling one or more sensors equipped therein.

The input unit 250 includes various input circuitry, such as, for example, and without limitation, a touch panel 252, a digital pen sensor 254, a key 256, or an ultrasonic input unit 258. The touch panel 252 may recognize a touch input in a manner of capacitive type, resistive type, infrared type, or ultrasonic type. Also, the touch panel 252 may further include a control circuit. The touch panel 252 may further include a tactile layer. In this case, the touch panel 252 may offer a tactile feedback to a user. The pen sensor 254 (e.g., a digital pen sensor), for example, may be implemented by using a method identical or similar to a method of receiving a touch input from the user, or by using a separate sheet for recognition. For example, a key pad or a touch key may be used similar to the keys 256. The ultrasonic input unit 258 enables the terminal to sense a sound wave by using a microphone 288 of the terminal through a pen generating an ultrasonic signal, and to identify data.

The display 260 (e.g., the display 160) includes a panel 262, a hologram 264, or a projector 266. The panel 262 may have a flexible, transparent or wearable form. The panel 262 may be formed of a single module with the touch panel 252. The hologram 264 may show a stereoscopic image in the air using interference of light. The projector 266 may project an image onto a screen, which may be located at the inside or outside of the electronic device 201. According to an embodiment of the present disclosure, the display 260 may further include a control circuit for controlling the panel 262, the hologram 264, and the projector 266.

The interface 270 may include various interface circuitry, such as, for example, and without limitation, an HDMI (high-definition multimedia interface) 272, a USB (universal serial Bus) 274, an optical interface 276, or a D-sub (D-subminiature) 278. The interface 270 may be contained, for example, in the communication interface 170 illustrated in FIG. 1. Additionally or alternatively, the interface 270 may include, for example, an MHL (mobile high-definition link) interface, an SD (secure digital) card/MMC (multi-media card) interface, or an IrDA (infrared data association) interface.

The audio module 280 may perform a conversion between sounds and electric signals. At least part of the audio module 280 may be contained, for example, in the input/output interface 150 illustrated in FIG. 1. The audio module 280 may process sound information inputted or outputted through a speaker 282, a receiver 284, an earphone 286, or a microphone 288.

The camera module 291 is a device capable of obtaining still images and moving images. According to an embodiment of the present disclosure, the camera module 291 may include at least one image sensor (e.g., a front sensor or a rear sensor), a lens, an ISP (image signal processor, not shown), or a flash (e.g., LED or xenon lamp, not shown).

The power management module 295 may manage electric power of the electronic device 201. The power management module 295 may include, for example, a PMIC (power management integrated circuit), a charger IC, or a battery charge gauge. The PMIC may be implemented by, for example, an IC or an SoC semiconductor. Charging methods may be classified into a wired charging method and a wireless charging method. A wireless charging type may include, for example, a magnetic resonance type, a magnetic induction type, or an electromagnetic type. Any additional circuit for wireless charging may be further used such as a coil loop, a resonance circuit, or a rectifier. The battery gauge may measure the residual charge amount of the battery 296 and a voltage, current or temperature in a charging process. The battery 296 may store or create electric power therein and supply electric power to the electronic device 201. The battery 296 may be, for example, a rechargeable battery or a solar battery.

The indicator 297 may show thereon a current status (e.g., a booting status, a message status, or a recharging status) of the electronic device 201 or of its part (e.g., the AP 210). The motor 298 may convert an electric signal into a mechanical vibration. The electronic device 201 may include a specific processor (e.g., GPU) for supporting a mobile TV. This processor may process media data that comply with standards of DMB (digital multimedia broadcasting), DVB (digital video broadcasting), or MediaFlo.

Each of the above-discussed elements of the electronic device disclosed herein may be formed of one or more components, and its name may vary according to the type of the electronic device. The electronic device disclosed herein may be formed of at least one of the above-discussed elements without some elements or with additional other elements. Some of the elements may be integrated into a single entity that still performs the same functions as those of such elements before integrated.

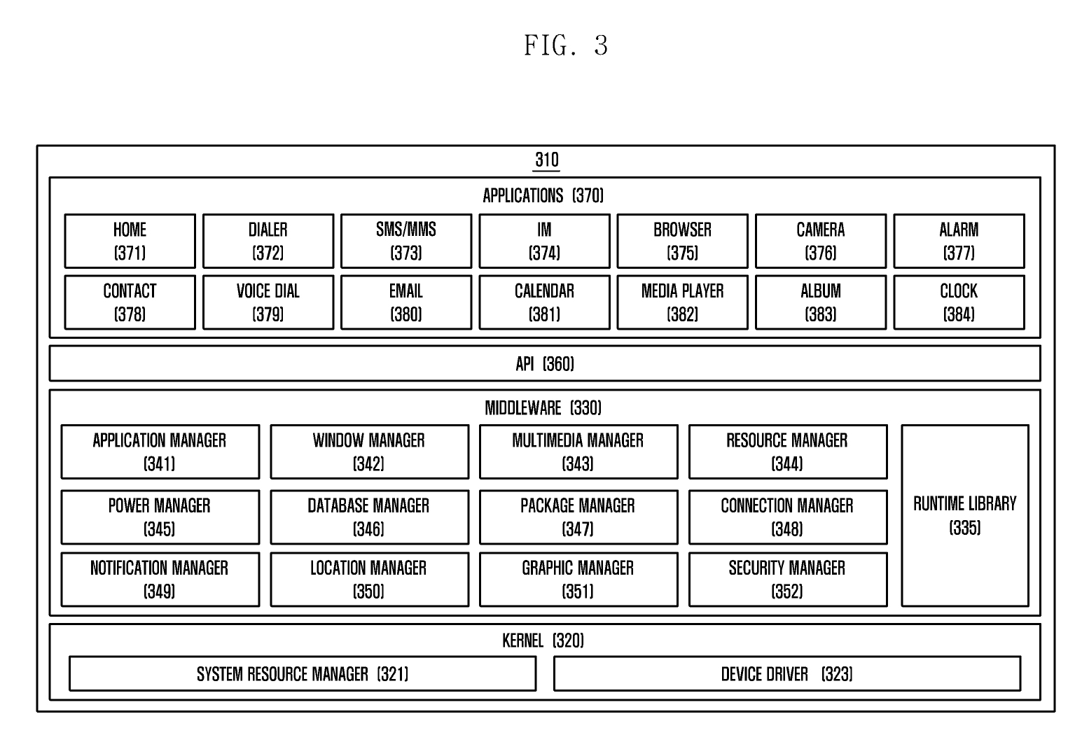

FIG. 3 illustrates an example program module, according to an example embodiment of the present disclosure.

Referring to FIG. 3, a program module 310 (e.g., the program 140) may include an operating system (OS) controlling resources related to the electronic device (e.g., the electronic device 101) and/or various applications (e.g., the application 147) that are driven on the operating system. The operating system may include, e.g., ANDROID®, iOS®, WINDOWS®, SYMBIAN®, TIZEN®, or SAMSUNG BADA®.

The program module 310 includes a kernel 320, middleware 330, an API 360, and/or an application 370. At least a part of the program module 310 may be preloaded on the electronic device or may be downloaded from the electronic device 104 or the server 106.

The kernel 320 (e.g., the kernel 141 of FIG. 1) may include, e.g., a system resource manager 321 and/or a device driver 323. The system resource manager 321 may perform control, allocation, or recovery of system resources and may include a process managing unit, a memory managing unit, and/or a file system managing unit. The device driver 323 may include, e.g., a display driver, a camera driver, a Bluetooth driver, a shared memory driver, a USB driver, a keypad driver, a Wi-Fi driver, an audio driver, or an inter-process communication (IPC) driver.

The middleware 330 may provide various functions to the application 370 through the API 360 so that the application 370 may efficiently use limited system resources in the electronic device or provide functions jointly required by applications 370. The middleware 330 (e.g., middleware 143) includes at least one of a runtime library 335, an application manager 341, a window manager 342, a multimedia manager 343, a resource manager 344, a power manager 345, a database manager 346, a package manager 347, a connection manager 348, a notification manager 349, a location manager 350, a graphic manager 351, and/or a security manager 352.

The runtime library 335 may include a library module used by a compiler to add a new function through a programming language while, e.g., the application 370 is being executed. The runtime library 335 may perform input/output management, memory management, and/or arithmetic functions.

The application manager 341 may manage the life cycle of at least one application of, e.g., the applications 370. The window manager 342 may manage graphical user interface (GUI) resources used on the screen. The multimedia manager 343 may determine formats necessary to play various media files and use a codec appropriate for a format to perform encoding or decoding on media files. The resource manager 344 may manage resources, such as source code of at least one of the applications 370, memory and/or storage space.

The power manager 345 may operate together with, e.g., a basic input/output system (BIOS) to manage battery or power and provide power information necessary for operating the electronic device. The database manager 346 may generate, search, and/or query a database to be used in at least one of the applications 370. The package manager 347 may manage installation or update of an application that is distributed in the form of a package file.

The connectivity manager 348 may manage wireless connectivity, such as, e.g., Wi-Fi or BT. The notification manager 349 may display or notify an event, such as an incoming message, appointment, and/or proximity notification without interfering with the user. The location manager 350 may manage location information on the electronic device. The graphic manager 351 may manage graphic effects to be offered to the user and their related user interface. The security manager 352 may provide various security functions necessary for system security and/or user authentication. When the electronic device (e.g., the electronic device 101) has telephony capability, the middleware 330 may further include a telephony manager for managing voice call and/or video call functions of the electronic device. The middleware 330 may include various functions of the above-described components. The middleware 330 may provide a specified module per type of operating system to provide a differentiated function. Further, the middleware 330 may dynamically omit some existing components or add new components.

The API 360 (e.g., the API 145) may be a set of, e.g., API programming functions and may have different configurations depending on operating systems. For example, in the case of Android or iOS, one API set may be provided per platform, and in the case of Tizen, two or more API sets may be offered per platform.

The application 370 (e.g., the application processor 147) includes one or more applications that may provide functions such as, e.g., a home 371, a dialer 372, a short message service (SMS)/multimedia messaging service (MMS) 373, an instant message (IM) 374, a browser 375, a camera 376, an alarm 377, a contact 378, a voice dial 379, an email 380, a calendar 381, a media player 382, an album 383, or a clock 384, a health-care (e.g., measuring the degree of workout or blood sugar level), and/or environmental information (e.g., provision of air pressure, moisture, or temperature information). The application 370 may include information exchanging application supporting information exchange between the electronic device (e.g., the electronic device 101) and an external electronic device 102 and 104. Examples of the information exchange application may include, but is not limited to, a notification relay application for transferring specific information to the external electronic device, or a device management application for managing the external electronic device. For example, a notification relay application may include a function for relaying notification information generated from other applications of the electronic device (e.g., the SMS/MMS application, the email application, the health-care application, or the environmental information application) to the external electronic devices 102 and 104. Further, the notification relay application may receive notification information from, e.g., the external electronic device and may provide the received notification information to the user. A device management application may perform at least some functions of the external electronic device 102 or 104 such as, for example, turning on/off the external electronic device (or some components of the external electronic device), control brightness (or resolution) of the display, etc. The device management application may manage (e.g., install, delete, or update) an application operating in the external electronic device or a service (e.g., call service or message service) provided from the external electronic device.

The application 370 may include an application (e.g., a health-care application) selected depending on the attribute (e.g., as an attribute of the electronic device, the type of electronic device is a mobile medical device) of the external electronic device 102 and 104. The application 370 may include an application received from the server 106 or electronic devices 102 and 104. The application 370 may include a preloaded application or a third party application downloadable from a server. The names of the components of the program module 310 may be vary depending on the type of operating system. At least a part of the program module 310 may be implemented in software, firmware, hardware, or in a combination of two or more thereof. At least a part of the programming module 310 may be implemented (e.g., executed) by e.g., a processor (e.g., the AP 210). At least a part of the program module 310 may include e.g., a module, a program, a routine, a set of instructions, a process, and the like for performing one or more functions.

The term “module” as used in this disclosure may refer to a certain unit that includes one of hardware (e.g., circuitry), software and firmware or any combination thereof. The term module may be interchangeably used with unit, logic, logical block, component, or circuit, for example. The module may be the minimum unit, or part thereof, which performs one or more particular functions. The module may be formed mechanically or electronically. For example, the module disclosed herein may include at least one of a processor (e.g., processing circuitry, CPU, etc.) ASIC (application-specific integrated circuit) chip, FPGAs (field-programmable gate arrays), and programmable-logic device, which have been known or are to be developed.

According to various embodiments disclosed herein, an electronic device may include a flexible housing including at least one connection unit, a communication unit located in the housing, at least one vibration motor located in the housing, at least one motion sensor location in the housing, a processor electrically coupled to at least one of the communication unit, the at least one vibration motor, and the at least one motion sensor, and a memory electrically coupled to the processor. In particular, the memory may store instructions causing, when executed, the processor to receive at least one signal through the communication unit, to execute a vibration notification, based on the received signal, to detect a motion of the device through the at least one motion sensor, to determine whether a state of the at least one connection unit is changed, to obtain information associated with a user's body part on which the device is worn, based on at least one of information associated with the detected motion and information associated with the changed state of the connection unit, and to perform at least one function, based on the obtained information.

In the electronic device according to various embodiments of this disclosure, the instructions may further cause the processor to determine whether the received signal is transmitted from another electronic device which is pairing with the device.

In the electronic device according to various embodiments of this disclosure, the instructions may further cause the processor to determine, through the at least one motion sensor, whether the motion exceeds a predetermined threshold, and if the motion exceeds the predetermined threshold, to receive sensing information through at least one of an altitude sensor, a gyro sensor and an acceleration sensor which are included in the device.

In the electronic device according to various embodiments of this disclosure, the instructions may further cause the processor to receive information about an altitude of the device at regular intervals through the altitude sensor.

In the electronic device according to various embodiments of this disclosure, the instructions may further cause the processor to receive information about an angle between the device and a reference plane at regular intervals through the gyro sensor.

In the electronic device according to various embodiments of this disclosure, the instructions may further cause the processor to receive information about a variation in velocity of the device at regular intervals through the acceleration sensor.

In the electronic device according to various embodiments of this disclosure, the instructions may further cause the processor to determine whether a length of the at least one connection unit is changed.

In the electronic device according to various embodiments of this disclosure, the instructions may further cause the processor to determine whether a variation in pressure is detected at the at least one connection unit.

In the electronic device according to various embodiments of this disclosure, the instructions may further cause the processor to trigger a heart rate monitor (HRM) sensor of the device, and to obtain the information associated with the user's body part through the HRM sensor.

In the electronic device according to various embodiments of this disclosure, the instructions may further cause the processor to change an operating state of at least one of a bone conduction unit speaker and a microphone which are included in the device.

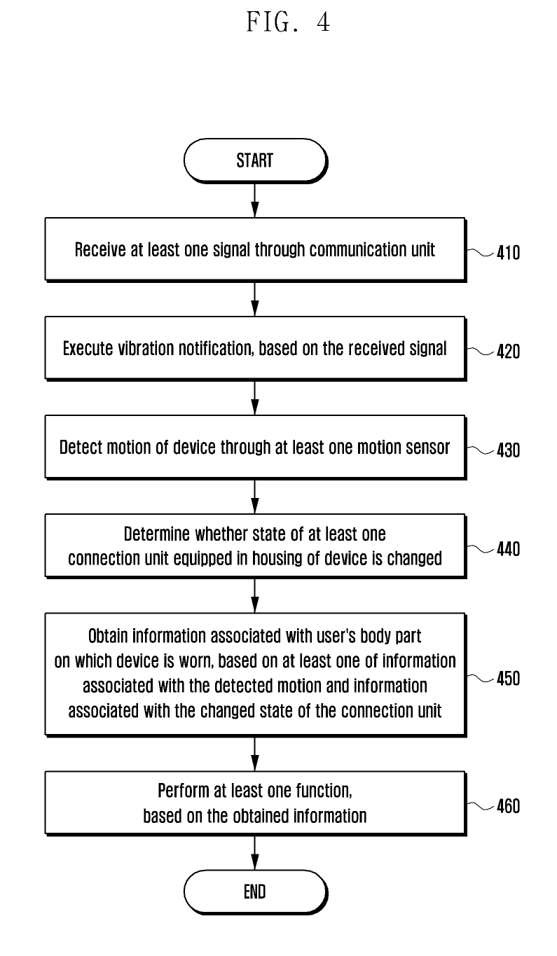

FIG. 4 illustrates a method for controlling a device by recognizing a motion of the device according to various embodiments.

According to an embodiment, at operation 410, at least one signal may be received through a communication unit equipped in the electronic device disclosed herein. The at least one signal may include a signal transmitted by any other electronic device. For example, when the electronic device is connected to any other electronic device such as a smart phone, a signal required for connection may be transmitted and received between the electronic device and the smart phone. Also, when the smart phone receives an incoming call, the electronic device may receive information about call reception from the smart phone through a signal. The communication unit may include, for example, the cellular module 221, the WiFi module 223, the Bluetooth module 225, the GNSS module 227 (e.g., a GPS module, a Glonass module, a Beidou module, or a Galileo module), the NFC module 228, and the RF module 229.

At operation 420, a notification may be executed, based on the signal received at operation 410. Namely, any notification unit equipped in the electronic device may create a notification corresponding to the signal received through the communication unit. For example, if a signal associated with the reception of an incoming call or message is received, a vibration motor included in the notification unit may perform a vibration notification. Through this vibration notification, a user can be aware of the occurrence of a specific event. The notification unit may include a speaker.

According to an embodiment, the electronic device may further include a display. A user can be aware of a visual-form notification through the display.

At operation 430, a motion of the electronic device may be detected through at least one motion sensor equipped in the electronic device. The electronic device disclosed herein may be attached to a part of user's body. Also, the electronic device may be set to perform different functions depending on the attached body parts even though the same event occurs. For example, the electronic device may be worn, as a bracelet, on user's wrist. In this case, when a notification associated with an incoming call is received, a user may detach the bracelet from the wrist and then wear it on the ear so as to use the electronic device as an earphone and a microphone. In this embodiment, in order to determine whether the electronic device is worn on the wrist or on the ear, a user's motion may be detected through the motion sensor equipped in the electronic device.

According to an embodiment, the electronic device has various kinds of sensors and, when a user takes a particular gesture, may perform a predetermined operation mapped to the user's gesture. A mapping relation between a gesture and a function may be predefined and stored. For example, in case of a gesture of shaking the electronic device worn on the wrist, the electronic device may perform a particular function mapped to the shaking gesture when a condition of shaking the electronic device for a given time is satisfied. Also, if a gesture of shaking a hand is entered in the electronic device, the electronic device may perform a function of rejecting an incoming call. Such a gesture, i.e., the motion of the electronic device, may be determined on the basis of the comparison of a current sensing value and a predetermined reference sensing value.

At operation 440, the electronic device may determine whether the state of at least one connection unit equipped in a housing of the electronic device is changed. The electronic device may have a transformable form so as to be worn on different parts of user's body. For example, the housing of the electronic device may be formed of a flexible material and also have a waterproof function in view of the wearability of the electronic device. For a transformable form, a telescopic shaft structure may be applied to the electronic device. The telescopic shaft structure is referred to as a shaft coupling structure that allows free retractability in the shaft direction. For example, if the end of one shaft formed of an outer tube having a polygonal cross-section is engaged with the end of the other shaft formed of an inner tube having a polygonal cross-section, it is possible to realize a shaft coupling for allowing a free slide in the shaft direction and also delivering a turning force. Namely, the electronic device may have an easily extendable and retractable structure. This structure may be applied individually to a plurality of connection units. Therefore, by checking a change in the state of at least one connection unit, it is possible to determine whether the device is attached to or detached from a corresponding body part.

At operation 450, based on information obtained by detecting the motion of the device at operation 430 or information obtained about a change in the state of the connection unit of the device at operation 440, the information associated with the body part of a user on which the device is worn may be obtained. For example, a user's motion may be detected through at least one motion sensor of the device at operation 430, and the device may obtain information about a movement from a user's wrist to a user's ear by analyzing the detected motion. Additionally, if the status of the connection unit of the device is changed to a first state at operation 440, the device may obtain information indicating that the device is detached from a user's wrist. Then if the status of the connection unit of the device is changed to a second state, the device may obtain information indicating that the device is worn on a user's ear. This information associated with user's body parts may include information about a location of the body part on which the device is worn or from which the device is detached.

At operation 460, the device may perform at least one function, based on the information obtained at operation 450. For example, when the device is worn on the wrist, the device may receive a signal associated with the reception of a call. In this case, the user can detach the device from the wrist and then wear the detached device on the ear. After it is recognized that the device is worn on the ear, the device may perform a call reception function. Also, the device may operate a speaker for receiving the other party's voice or a microphone for transmitting a user's voice.

The above-discussed embodiments are offered as examples to help reader's understanding. Therefore the operations of the electronic device are not limited to such embodiments.

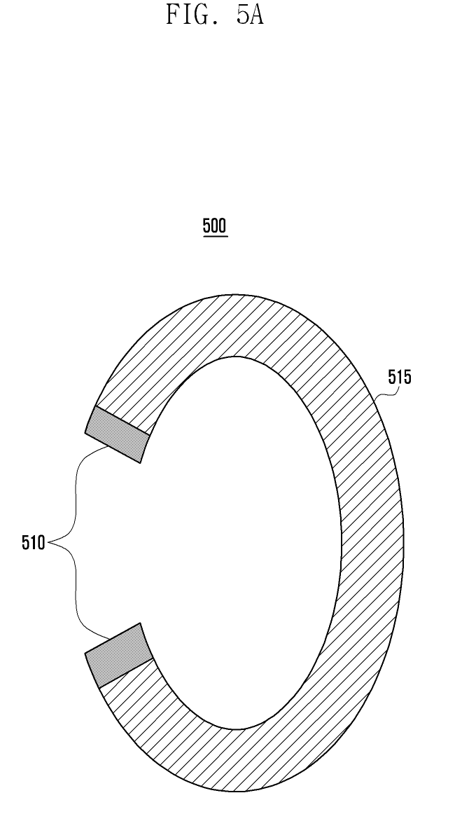

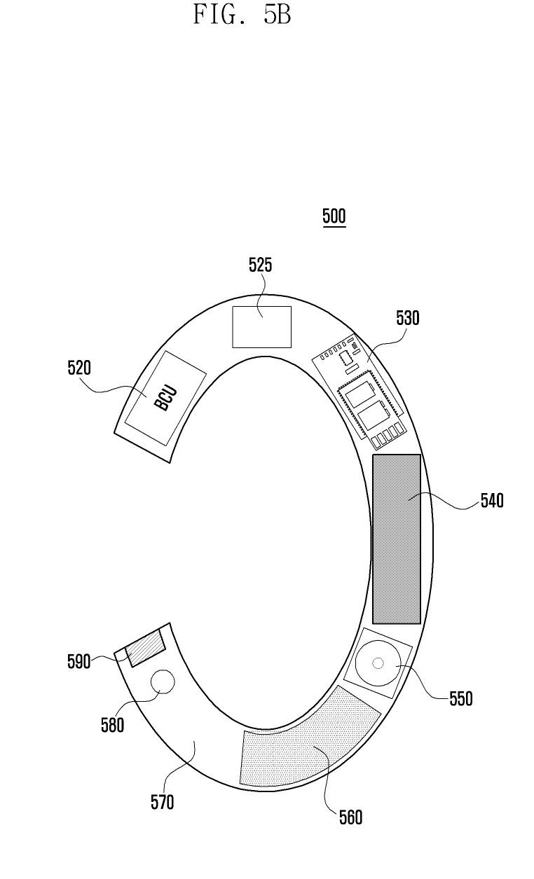

FIGS. 5A and 5B illustrate a structure of an electronic device according to various embodiments.

FIG. 5A shows an appearance of the electronic device 500. The electronic device 500 according to an embodiment may have both ends 510 which are separated from each other. Each end 510 of the device 500 may have a cap for finishing a housing 515 of the device 500. The device 500 may have a transformable form to be worn on a plurality of body parts. For example, the housing of the device 500 may be formed of a flexible material and also have a waterproof function in view of the wearability of the electronic device. Silicone, rubber or any equivalent may be used as an outer material of the housing 515.

The inner part of the housing 515 may have at least one connection unit that allows a change in length or bend of the device 500. The connection unit may have a telescopic shaft structure such that the device 500 can be easily extended or retracted in length or easily bent or straightened.

FIG. 5B shows an internal structure of the electronic device 500. Referring to FIG. 5B, the electronic device 500 may include a speaker 520, a circuit unit 525, a communication unit 530, a power supply unit 540, a vibration motor 550, a sensor unit 560, a connection unit 570, an input unit 580, and an input/output terminal 590.

The speaker 520 may include a bone conduction unit (BCU) speaker. The BCU speaker 520 may use a delivery of a sound through bones. A person can perceive and hear a sound delivered through a body (especially, a vibration of bones and blood). Therefore, using the BCU speaker 520, the user can perceive a sound when a vibration of a bone conduction vibrator located near the skull reaches the internal ear through the cranial bone. Although the BCU speaker 520 is discussed in this embodiment, any other speaker such as a sound speaker or a piezo-type speaker may be used alternatively.

The circuit unit 525 is an element for electrically coupling various modules of the device 500. The circuit unit 525 may include a camera module, an audio module, or any other necessary module, and also have a memory, a processor, or the like.

The communication unit 530 may include, for example, the cellular module 221, the WiFi module 223, the Bluetooth module 225, the GNSS module 227 (e.g., a GPS module, a Glonass module, a Beidou module, or a Galileo module), the NFC module 228, and the RF module 229. The device 500 may be connected to any other electronic device through the communication unit 530. Additionally, using the communication unit 530, the device 500 may receive a signal from any external entity and transmit data, created therein, to any other electronic device. For example, the device 500 may perform pairing with any other electronic device through the WiFi module 223 or the Bluetooth module 225.

The power supply unit 540 may include at least one battery (not shown), a power supply circuit, a battery charging circuit, or the like disposed in the housing of the device 500. The power supply unit 540 may supply electric power to the device 500. Namely, the power supply unit 540 may receive electric power from an external power source (not shown) through a cable connected with the input/output terminal 590 and then supply the received power to each module or battery of the device 500. In addition, the power supply unit 540 may receive electric power from the external power source through wireless charging technology and then supply the received power to each module or battery of the device 500.

The vibration motor 550 may convert an electric signal into a mechanical vibration and thus generate a vibration or haptic effect.

The sensor unit 560 may include one or more sensor modules, each of which measures a physical quantity or senses an operating state of the device 500 and then converts the measured or sensed information into an electric signal. For example, the sensor modules may include a motion sensor and further include, as shown in FIG. 2, at least one of the gesture sensor 240A, the gyro sensor 240B, the atmospheric pressure sensor 240C, the magnetic sensor 240D, the acceleration sensor 240E, the grip sensor 240F, the proximity sensor 240G, the color sensor 240H (e.g., the RGB sensor), the biometric sensor 2401, the temperature/humidity sensor 240J, the illuminance sensor 240K, and the UV sensor 240M. Additionally or alternatively, the sensor module may include, for example, an E-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module may further include a control circuit for controlling the above sensor(s). In a certain embodiment, the device 500 may further include a processor, as a part of or separately from the processor 210, configured to control the sensor module. This processor may control the sensor module while the processor 210 is in a sleep state. In another embodiment, the device 500 may include a heart rate monitor (HRM) sensor and, through the HRM sensor, obtain information associated with the body part to which the device 500 is attached.

The device 500 may include at least one connection unit 570. As discussed above, the telescopic shaft structure may be applied to the connection unit 570. The telescopic shaft structure allows the device 500 to be easily extended or retracted in length or easily bent or straightened. The connection unit 570 may have an additional sensor through which the device may determine whether the state of the connection unit is changed.

The input unit 580 may include a microphone. According to an embodiment, when a call is performed through the device 500, the device 500 may be worn on user's ear to allow the user to talk through the microphone.

The input/output terminal 590 may include a charging terminal, a universal serial bus (USB), or the like. The device 500 may be connected with any other electronic device through interfaces equipped in the input/output terminal 590.

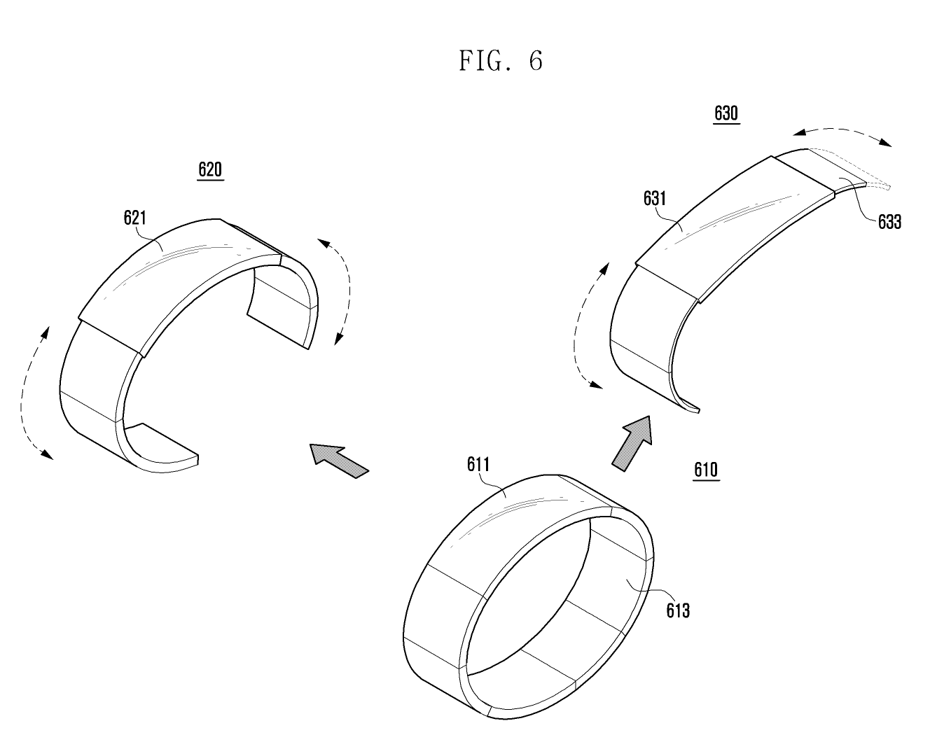

FIG. 6 illustrates a connection unit of an electronic device according to various embodiments.

As discussed above, the electronic device 500 may include the housing 515 formed of a flexible material and the connection unit(s) 570. Now referring to FIG. 6, the device 610, 620 or 630 may include a fixed unit 611, 621 or 631 and at least one connection unit 613, 623 or 633. The connection unit formed of the telescopic shaft structure may be easily changed in length from at least one of both sides of the fixed unit. If there are two or more connection units, the device may operate such connection units independently.

In the device 610 having the fixed unit 611 and the connection units 613, the connection units 613 may be extended respectively in opposite curved directions from both sides of the fixed unit 611 and thus form a band shape.

As shown in the device 620, the connection units 623 may be extended respectively to similar lengths toward opposite directions. Also, as shown in the device 630, the connection units 633 may be extended respectively to different lengths toward opposite directions.

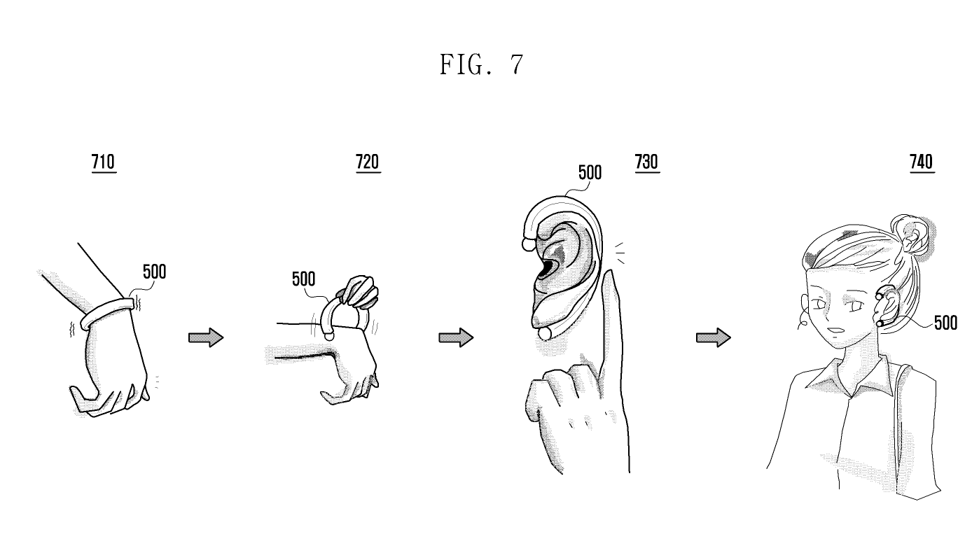

FIG. 7 illustrates an operation method of an electronic device according to various embodiments.

As indicated by a reference numeral 710, the electronic device 500 may be worn on a user's wrist. Then the device 500 may be used as a state of connection with any other electronic device. For example, the device 500 may receive a signal from the connected other electronic device. Then, based on the received signal, the device 500 may perform a notification function. Namely, the notification unit equipped in the device 500 may produce a notification corresponding to the signal received through the communication unit of the device 500. For example, when a signal associated with the reception of an incoming call or message is received, a vibration motor included in the notification unit may execute a vibration notification. Through this vibration notification, a user can be aware of the occurrence of a specific event.

The reference numeral 710 shows a situation in which any other electronic device connected with the device 500 receives an incoming call and then transmits a signal associated with the reception of a call to the device 500. In this situation, at least one notification including the above-mentioned vibration notification may be produced in the device 500.

A user may receive an incoming call by using the device 500. For this, as indicated by a reference numeral 720, the user may detach the device 500 from the wrist. At this time, the band shape of the device 500 may be changed to any other suitable form for being detachable through the connection unit(s) of the device. For example, the device 500 may be straightened for easy detachment.

Then, as indicated by a reference numeral 730, the detached device 500 may be moved to and worn on a user's ear. At this time, the device 500 having the connection unit(s) may be adjusted to a suitable size for the ear. Also, based on the transformed form of the connection unit, the device 500 may recognize that the device 500 is worn on the ear. Alternatively, based on a user's motion detected during a movement from the wrist to the ear, the device 500 may recognize that the device is worn on the ear. After this recognition, the device 500 may perform a call reception function automatically. Alternatively, depending on user's setting, the device 500 may perform a call reception function in response to any input entered to at least one input unit (not shown) of the device.

While performing the call reception function, the device 500 may trigger at least one necessary element thereof. As indicated by a reference numeral 740, the device 500 may operate a speaker and a microphone so as to support a user's call. According to an embodiment, the speaker may include the above-discussed BCU speaker.

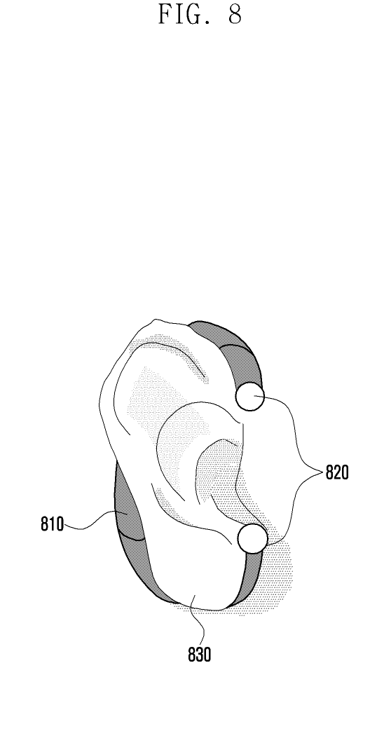

FIG. 8 illustrates an example of using an electronic device according to various embodiments.

According to an embodiment, the device 810 may be worn on a user's ear 830. The device 810 may include at least one bendable and length-adjustable connection unit. Thus, the device 810 may have a flexible structure.

Both ends of the device 810 may have an additional member 820 to be used for attaching the device to a user's body part. This member 820 may be formed of magnet or any other equivalent suitable for fixedly connecting both ends of the device 810. Also, this member 820 may be covered with a soft cap for user's convenience.

According to various embodiments disclosed herein, a method for providing a user interaction at an electronic device may include operations of receiving at least one signal through a communication unit of the device, executing a vibration notification, based on the received signal, detecting a motion of the device through at least one motion sensor of the device, determining whether a state of at least one connection unit equipped in a housing of the device is changed, obtaining information associated with a user's body part on which the device is worn, based on at least one of information associated with the detected motion and information associated with the changed state of the connection unit, and performing at least one function, based on the obtained information.

In the method according to various embodiments of this disclosure, the operation of receiving the signal may includes determining whether the received signal is transmitted from another electronic device which is pairing with the device.

In the method according to various embodiments of this disclosure, the operation of detecting the motion of the device may include determining, through the at least one motion sensor, whether the motion exceeds a predetermined threshold, and if the motion exceeds the predetermined threshold, triggering at least one of an altitude sensor, a gyro sensor and an acceleration sensor which are included in the device.

In the method according to various embodiments of this disclosure, the operation of detecting the motion of the device may further include measuring a variation in altitude of the device at regular intervals through the altitude sensor.

In the method according to various embodiments of this disclosure, the operation of detecting the motion of the device may further include measuring an angle between the device and a reference plane at regular intervals through the gyro sensor.

In the method according to various embodiments of this disclosure, the operation of detecting the motion of the device may further include measuring a variation in velocity of the device at regular intervals through the acceleration sensor.

In the method according to various embodiments of this disclosure, the operation of determining whether the state of the at least one connection unit is changed may include determining whether a length of the at least one connection unit is changed.

In the method according to various embodiments of this disclosure, the operation of determining whether the state of the at least one connection unit is changed may include determining whether a variation in pressure is detected at the at least one connection unit.

In the method according to various embodiments of this disclosure, the operation of obtaining the information associated with the user's body part may include triggering a heart rate monitor (HRM) sensor of the device, and obtaining the information associated with the user's body part through the HRM sensor.

In the method according to various embodiments of this disclosure, the operation of performing the at least one function may include changing an operating state of at least one of a bone conduction unit speaker and a microphone which are included in the device.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A method for providing a user interaction at an electronic device, the method comprising operations of:
receiving at least one signal through a communication unit of the device;
executing a vibration notification, based on the received signal;
detecting a motion of the device through at least one motion sensor of the device;
determining whether a state of at least one connection unit equipped in a housing of the device is changed;
obtaining information associated with a user's body part on which the device is worn, based on at least one of information associated with the detected motion and information associated with the changed state of the connection unit; and
performing at least one function, based on the obtained information.

2. The method of claim 1, wherein receiving the at least one signal includes determining whether the received signal is transmitted from another electronic device which is pairing with the device.

3. The method of claim 1, wherein detecting the motion of the device includes:
determining, through the at least one motion sensor, whether the motion exceeds a predetermined threshold; and
if the motion exceeds the predetermined threshold, triggering at least one of an altitude sensor, a gyro sensor, or an acceleration sensor which are included in the device.

4. The method of claim 3, wherein detecting the motion of the device further includes measuring a variation in altitude of the device at regular intervals through the altitude sensor.

5. The method of claim 3, wherein detecting the motion of the device further includes measuring an angle between the device and a reference plane at regular intervals through the gyro sensor.

6. The method of claim 3, wherein detecting the motion of the device further includes measuring a variation in velocity of the device at regular intervals through the acceleration sensor.

7. The method of claim 1, wherein determining whether the state of the at least one connection unit is changed includes determining whether a length of the at least one connection unit is changed.

8. The method of claim 1, wherein determining whether the state of the at least one connection unit is changed includes determining whether a variation in pressure is detected at the at least one connection unit.

9. The method of claim 1, wherein obtaining the information associated with the user's body part includes:
triggering a heart rate monitor (HRM) sensor of the device; and
obtaining the information associated with the user's body part through the HRM sensor.

10. The method of claim 1, wherein performing the at least one function includes changing an operating state of at least one of a bone conduction unit speaker or a microphone which are included in the device.

11. An electronic device comprising:
a flexible housing including at least one connection unit;
a communication unit located in the housing;
at least one vibration motor located in the housing;
at least one motion sensor located in the housing;
a processor electrically coupled to at least one of the communication unit, the at least one vibration motor, and the at least one motion sensor; and
a memory electrically coupled to the processor,
wherein the memory stores instructions causing, when executed, the processor to:
receive at least one signal through the communication unit,
execute a vibration notification, based on the received signal,
detect a motion of the device through the at least one motion sensor,
determine whether a state of the at least one connection unit is changed,
obtain information associated with a user's body part on which the device is worn, based on at least one of information associated with the detected motion and information associated with the changed state of the connection unit, and
perform at least one function, based on the obtained information.

12. The electronic device of claim 11, wherein the instructions further cause the processor to determine whether the received signal is transmitted from another electronic device which is paired with the device.

13. The electronic device of claim 11, wherein the instructions further cause the processor to:
determine, through the at least one motion sensor, whether the motion exceeds a predetermined threshold; and
if the motion exceeds the predetermined threshold, receive sensing information through at least one of an altitude sensor, a gyro sensor, or an acceleration sensor which are included in the device.

14. The electronic device of claim 13, wherein the instructions further cause the processor to receive information about an altitude of the device at regular intervals through the altitude sensor.

15. The electronic device of claim 13, wherein the instructions further cause the processor to receive information about an angle between the device and a reference plane at regular intervals through the gyro sensor.

16. The electronic device of claim 13, wherein the instructions further cause the processor to receive information about a variation in velocity of the device at regular intervals through the acceleration sensor.

17. The electronic device of claim 11, wherein the instructions further cause the processor to determine whether a length of the at least one connection unit is changed.

18. The electronic device of claim 11, wherein the instructions further cause the processor to determine whether a variation in pressure is detected at the at least one connection unit.

19. The electronic device of claim 11, wherein the instructions further cause the processor to:
trigger a heart rate monitor (HRM) sensor of the device; and
obtain the information associated with the user's body part through the HRM sensor.

20. The electronic device of claim 11, wherein the instructions further cause the processor to:
change an operating state of at least one of a bone conduction unit speaker or a microphone which are included in the device.
